(19) United States
(12) Patent Application Publication
(10) Pub. No.: US 2020/0306043 A1
(43) Pub. Date: Oct. 1, 2020

(54) VALVE TRANSLOCATION DEVICE AND METHOD FOR THE TREATMENT OF FUNCTIONAL VALVE REGURGITATION

(71) Applicants: University of Maryland, Baltimore, Baltimore, MD (US); University of Maryland Medical System LLC, Baltimore, MD (US)

(72) Inventors: James Gammie, Stevenson, MD (US); Rachael Quinn, Abingdon, MD (US); Chetan Pasrija, Gaithersburg, MD (US)

(21) Appl. No.: 16/761,181

(22) PCT Filed: Nov. 5, 2018
(86) PCT No.: PCT/US2018/059253

§ 371 Date: May 1, 2020

Related U.S. Application Data
(60) Provisional application No. 62/581,085, filed on Nov. 3, 2017.

(51) Int. Cl. A61F 2/24

(52) U.S. Cl. CPC A61F 2/2454; A61F 2/2445; A61B 8/0883; A61F 2250/001; A61F 2220/0075; A61F 2230/0065

(57) ABSTRACT

The present invention provides devices for treating functional mitral regurgitation and methods of use thereof. The devices translocate a subject's mitral valve in an apical direction. The devices thereby treats mitral regurgitation while preserving a subject's original mitral valve and chordae tendinae.

### CROSS-REFERENCE TO RELATED APPLICATIONS

This application claims priority to U.S. Provisional Patent Application No. 62/581,085, filed Nov. 3, 2017, the contents of which are incorporated by reference herein in its entirety.

### BACKGROUND OF THE INVENTION

There is presently no reliable, durable mitral valve repair option for patients with functional mitral regurgitation (FMR). In patients with FMR, the mitral valve is usually normal, but left ventricular dysfunction (due to coronary artery disease, idiopathic myocardial disease, or nonischemic cardiomyopathy) is present. The abnormal and dilated left ventricle causes papillary muscle displacement, which results in leaflet tethering with associated annular dilation that prevents coaptation (generally defined as abutment of the edges of the two mitral valve leaflets). Thus, fundamental geometric issues of FMR include annular dilation, annular flattening, leaflet tethering, and increased interpapillary distances.

Although restrictive mitral annuloplasty (RMA) is usually initially effective in abrogating mitral regurgitation, there is clear data that these repairs are not as durable as replacing the mitral valve with a prosthetic (tissue or mechanical) valve. RMA involves suturing a semi-rigid ring around the perimeter of the mitral valve (the annulus) to decrease the area of the mitral orifice and increase the amount of coaptation of the two leaflets. The frequent progressive ineffectiveness of RMA is generally due to continued adverse remodeling and enlargement of the left ventricle, with continued geometric distortion—including continued restriction of the leaflets into the ventricular cavity with resulting failure of coaptation.

For example, in the ACORN trial (see J Thorac Cardiovasc Surg. 2011 September; 142(3):569-74) that included mostly patients with idiopathic FMR, the recurrence rate of severe mitral regurgitation (MR) was 19 percent at 5 years. Recent data from a randomized trial that compared repair and replacement of the mitral valve for severe FMR demonstrated that nearly 60 percent of patients with mitral valve repairs or replacements suffered recurrence of moderate or greater MR at 2 years. Importantly, the group of patients with recurrence also showed less favorable ventricular reverse remodeling (i.e. they had bigger ventricles) compared to a repair group that had durable treatment of MR. Fundamentally, a restrictive mitral annuloplasty does not leave an adequate surface area for coaptation. A variety of techniques have been tried to repair FMR in a more durable fashion than current techniques, but none have had widespread adoption or success.

While replacing the mitral valve with a prosthetic valve is the most durable current technique, prosthetic valves have significant downsides, including risks of thromboembolism, prosthetic valve infection, degeneration of bioprostheses, mandatory anticoagulation of mechanical valves, and a higher perioperative mortality risk. While there are clear benefits to mitral valve repair compared to replacement for patients with degenerative mitral valve disease, annuloplasty insertion for treating FMR is associated with a very high rate of early recurrence of mitral regurgitation (e.g., 58% at two years in a randomized CTSN trial, NEJM 2016).

Common techniques often use a MitraClip®, and are based on a surgical approach (the “Alfieri” stitch) that is known to be only variably effective. The MitraClip® procedure involves placing a Dacron®-covered titanium clip such that the middle portion of the anterior and posterior leaflets are joined, which forms the mitral valve into a “double orifice” valve. Results from treatment of FMR with the MitraClip® have been suboptimal and a substantial number of patients have either residual or recurrent mitral regurgitation.

Therefore, there is a need in the art for improved devices and methods for treating functional mitral regurgitation. The present invention addresses this need.

### SUMMARY OF THE INVENTION

In one aspect, the present invention provides a translocation collar device comprising: a substantially ring-shaped band of material having an annular edge, an apical edge opposite from the annular edge, and a width in between; wherein the collar device is attachable to a circumferentially separated valve such that the annular edge is attached to a valve annulus and the apical edge is attached to a valve perimeter to translocate the valve in an apical direction.

In one embodiment, the annular edge has a diameter between about 20 mm and 60 mm. In one embodiment, the apical edge has a diameter between about 5 mm and 15 mm. In one embodiment, the width is between about 5 mm and 15 mm. In one embodiment, the width is biased such that the annular edge and the apical edge are separated by a variable distance.

In one embodiment, the device is constructed from a length of material having an arc shape, with a first end, a second end, an outer edge having a length equal to a circumference of the annular edge, and an inner edge having a length equal to a circumference of the apical edge, such that the first end and the second end are joinable together to form a substantially ring-shaped band.

In one embodiment, the device further comprises one or more concentric folds aligned in parallel with the annular edge and the apical edge, such that the width is variable. In one embodiment, the width is fixable by applying one or more sutures or adhesives to the one or more concentric folds.

In one embodiment, the device further comprises one or more annuloplasty rings attached to the annular edge, the apical edge, or both. In one embodiment, the device further comprises one or more cuffs attached to the annular edge, the apical edge, or a position in between.

In one embodiment, the material is selected from the group consisting of: polymer, fabrics, plastics, metals, autograft tissue, allograft tissue, xenograft tissue, and engineered tissue constructs.

In another aspect, the present invention relates to a method of translocating a valve, the method comprising the steps of: providing a translocation collar device having a ring-like shape with an annular edge, an apical edge, and a width in between; forming a circumferential incision around a perimeter of a valve to separate a valve annulus from a valve perimeter; circumferentially attaching the annular edge of the collar device to the valve annulus; and circumferentially attaching the apical edge of the collar device to the valve perimeter.

In one embodiment, the translocation collar device is sized to fit the valve annulus and valve perimeter by measuring the dimensions of the valve annulus and valve perimeter. In one embodiment, the translocation collar device is sized to fit the valve annulus and valve perimeter by performing and measuring the dimensions of a 3D echocardiogram of a heart containing the valve.

In one embodiment, the circumferential incision is formed while keeping the valve and associated structures intact, the associated structures including leaflets, commissures, chordae tendinae, and papillary muscles.

In one embodiment, the annular edge and the apical edge of the collar device are attached after the circumferential incision is fully formed and the valve annulus is completely separated from the valve perimeter. In one embodiment, the annular edge and the apical edge of the collar device are attached as the circumferential incision is being formed and the valve annulus is partially separated from the valve perimeter.

In another aspect, the present invention relates to a valve translocation kit, comprising: at least one translocation collar device having a ring-like shape with an annular edge, an apical edge, and a width in between; and one or more suture threads, suture pledgets, forceps, scissors, scalpels, and combinations thereof.

In one embodiment, the kit further comprises one or more circumferential bands, each circumferential band configured to wrap around papillary muscles connected to a valve. In one embodiment, the kit further comprises one or more tethers, each tether configured to attach to the apical edge of device at a first end and papillary muscles connected to a valve at a second end.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1A and FIG. 1B depict the anatomy of a left atrium, left ventricle, and mitral valve. FIG. 1C depicts an exemplary translocation collar device and the implantation of the translocation collar device around the mitral valve to displace it towards the apex of the heart.

FIG. 2A and FIG. 2B depict exemplary translocation collar devices having biased heights.

FIG. 3A through FIG. 3C depict exemplary translocation collar devices having annuloplasty rings.

FIG. 4A through FIG. 4C depict exemplary translocation collar devices having adjustable heights.

FIG. 5A through FIG. 5C depict exemplary translocation collar devices having sewing cuffs.

FIG. 6 depicts an exemplary implanted translocation collar device with the addition of a circumferential band around the chordae tendinae and papillary muscles.

FIG. 7 depicts an exemplary implanted translocation collar device with the addition of supplementary tethers connected to the papillary muscles.

FIG. 8 is a flowchart depicting an exemplary method of implanting a translocation collar device.

FIG. 9A through FIG. 9E illustrate the steps of implanting an exemplary translocation collar device around a mitral valve.

FIG. 10 depicts common surgical approaches to treat functional mitral regurgitation, each with its own drawbacks.

FIG. 11A and FIG. 11B depict the implantation of an exemplary translocation collar device into an excised porcine heart. The collar is sewn circumferentially to the mitral valve (i.e., first/distal suture line). After distal suture line is complete, a standard mitral annuloplasty ring is placed around the collar at the level of the suture line, it is next sewn in place. The annuloplasty ring serves to stabilize and fix the perimeter of the mitral valve in an optimal configuration. In the final result, the collar is in place and the mitral valve is translocated toward the ventricle.

FIG. 12 depicts three prototype translocation collar devices having varying widths.

FIG. 13A and FIG. 13B depict the results of an in vivo swine analysis comparing the effects of implanting a prototype translocation collar device on mitral valve coaptation.

FIG. 14A and FIG. 14B depict a modified fabrication process to create a prototype translocation collar device having a steeper angle and sized to the dimensions of a recipient's annulus.

FIG. 15A and FIG. 15B depict an implanted translocation collar device having an upper diameter of 40 mm and a lower diameter of 28 mm.

FIG. 16A and FIG. 16B depict an implanted translocation collar device having an upper diameter of 32 mm and a lower diameter of 28 mm.

FIG. 17A and FIG. 17B depict the use of non-locking sutures (FIG. 17A) and the use of locking sutures (FIG. 17B) to prevent crimping.

FIG. 18A through FIG. 18D depict a sequence of implanting a prototype translocation collar device having 2 mm upper and lower tabs for horizontal mattress sutures.

### DETAILED DESCRIPTION

The present invention provides devices for treating functional mitral regurgitation and methods of use thereof. The devices translocate a subject's mitral valve in an apical direction. The devices thereby treats mitral regurgitation while preserving a subject's original mitral valve and chordae tendinae.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments therebetween. This applies regardless of the breadth of the range.

### Translocation Collar Device

The present invention provides devices and techniques for durable and reasonably adaptable valve translocation to repair and treat regurgitation. The device translocates a subject's mitral valve in the ventricle, positioning the mitral valve toward the apex of the subject's heart to compensate for the fundamental geometric issues of FMR, such as annular dilation, annular flattening, leaflet tethering, and increased interpapillary distances. The device is configured to decrease the amount of tethering of the patient's mitral valve leaflets to increase the coaptation surface area between the two mitral valve leaflets. The device preserves the original mitral valve and chordae tendinae in an intact manner. Therefore, the device increases the likelihood of a durable repair and an effective and lasting treatment of FMR. In addition, the device addresses flattening of the annulus and annular dilation.

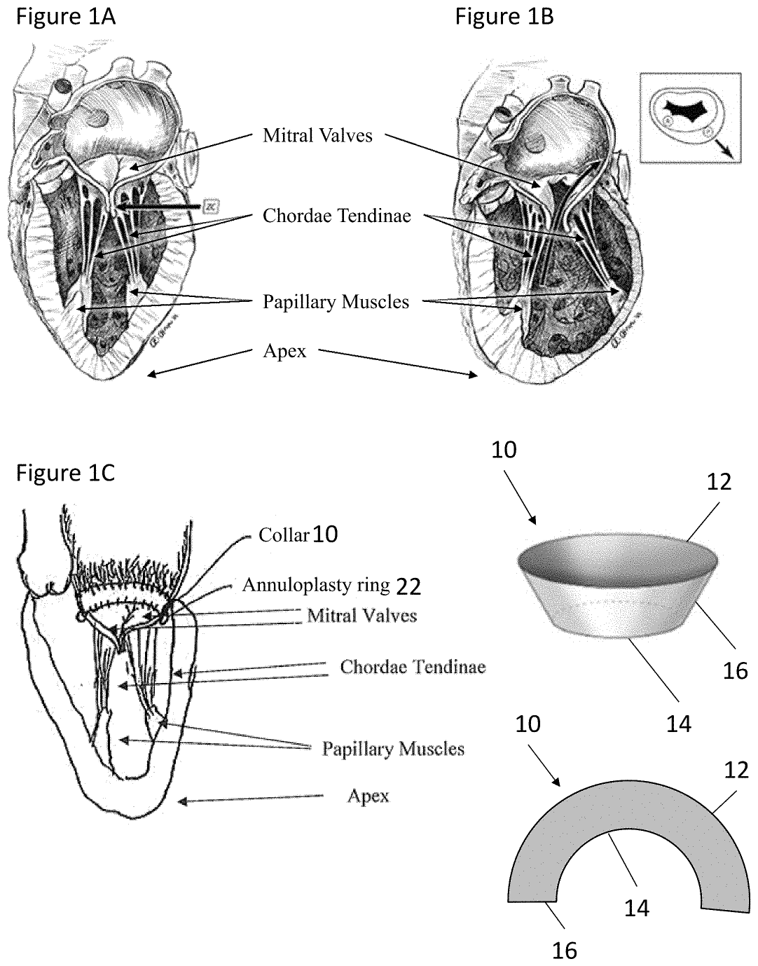

Referring now to FIG. 1C, an exemplary translocation collar device 10 is depicted. Device 10 comprises a ring-like band of material having an exterior, an interior, an annular edge 12, an apical edge 14, and a width 16 in between. Annular edge 12 is configured and shaped to attach to a subject's mitral annulus, and apical edge 14 is configured and shaped to attach to a subject's translocated mitral valve. Annular edge 12 forms an annular opening having an annular opening diameter. Apical edge 14 forms an apical opening that forms a neoannulus and has an apical opening diameter. Contemplated dimensions for device 10 include but are not limited to annular edge 12 having a diameter between about 20 mm and 60 mm, apical edge 14 having a diameter between about 5 mm and 15 mm, and width 16 between about 5 mm and 15 mm. The annular edge opening and the apical edge opening together form a valvular aperture. In some embodiments, the annular opening diameter is larger than the apical diameter opening, and the apical opening and the annular opening are each planar, such that device 10 has a substantially truncated (e.g., frustum conical-like) conical shape.

In one embodiment, annular edge 12 is sized to have substantially the same diameter as the diameter of a subject's mitral annulus in a dilated condition, while apical edge 14 is sized to have substantially the same diameter and shape of a subject's normal mitral valve in an undilated condition. In some embodiments, device 10 is formed from a length of material having width 16 and an arc-shape, wherein an outer edge has a length equal to the circumference of annular edge 12 and an inner edge has a length equal to the circumference of apical edge 14, such that the thin band of material can be joined end-to-end to form the substantially truncated conical shape of device 10.

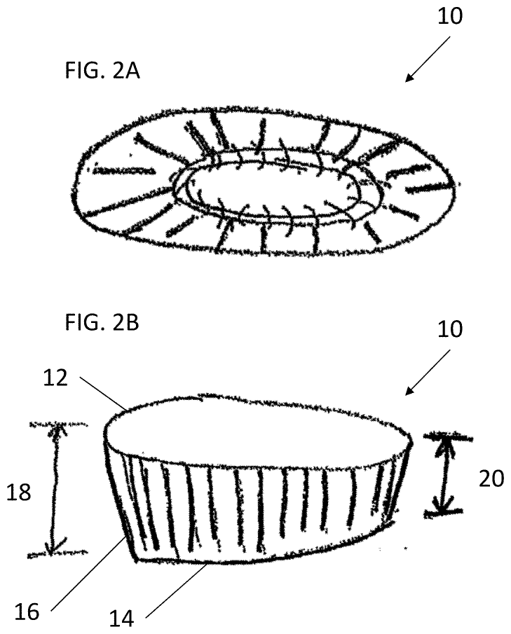

Referring now to FIG. 2A and FIG. 2B, in some embodiments device 10 has an oblique or slanted truncated cone-like shape, in which annular edge 12 and apical edge 14 are not generally concentric. For example, while in some embodiments annular edge 12 and apical edge 14 can be in parallel alignment, in other embodiments annular edge 12 and apical edge 14 can have an angle difference that is generally between about 30 degrees and 80 degrees, such as an angle difference between about 60 degrees and 70 degrees. Device 10 has a variable width 16, thereby forming a first height 18 and a second height 20, wherein a first height 18 can be greater, such as in a posteromedial segment. While a symmetrical device 10 can be advantageous for subjects having global left ventricular dysfunction that require symmetric displacement of the mitral valve into the ventricle, a device 10 having an oblique or slanted shape can be advantageous to treat a subject's particular condition, such as a greater height at a posteromedial portion for inferior ischemic myopathy.

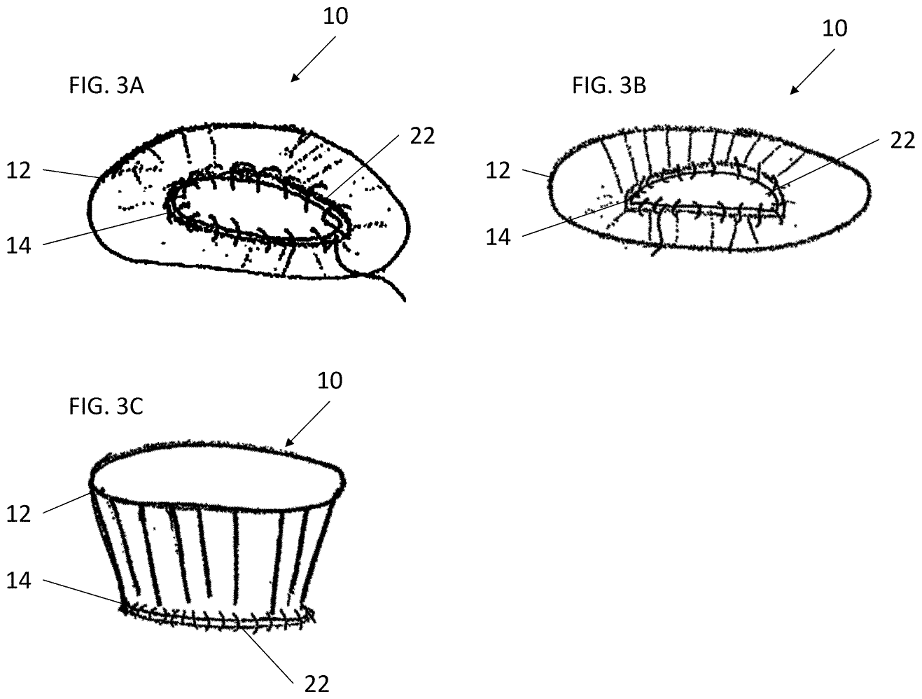

Referring now to FIG. 3A through FIG. 3C, in some embodiments device 10 can further include an annuloplasty ring 22. Annuloplasty ring 22 can be attached to the interior or exterior of annular edge 12, apical edge 14, or both. In some embodiments, annuloplasty ring 22 can have an asymmetrical shape or non-circular shape sized to fit the anatomy of a subject, as would be understood by those skilled in the art. Annuloplasty ring 22 can have any typical size, such as a size 26, 28, or 30 annuloplasty ring or the like.

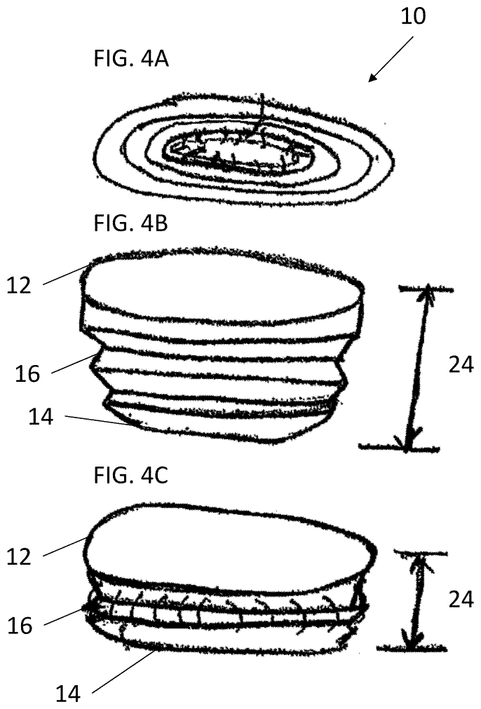

Referring now to FIG. 4A through FIG. 4C, in some embodiments device 10 can further include concentric folding aligned in parallel with annular edge 12 and apical edge 14 along width 16, providing device 10 with a customizable height 24. Device 10 can be used with unaltered concentric folding to provide a variable range of movement after implantation. A variable range of movement can be advantageous by permitting device 10 to adapt to varying heart rates. Device 10 can also have height 24 customized to the dimensions of a subject by placing a suture or adhesive along the concentric folding, thereby fixing height 24 (FIG. 4C).

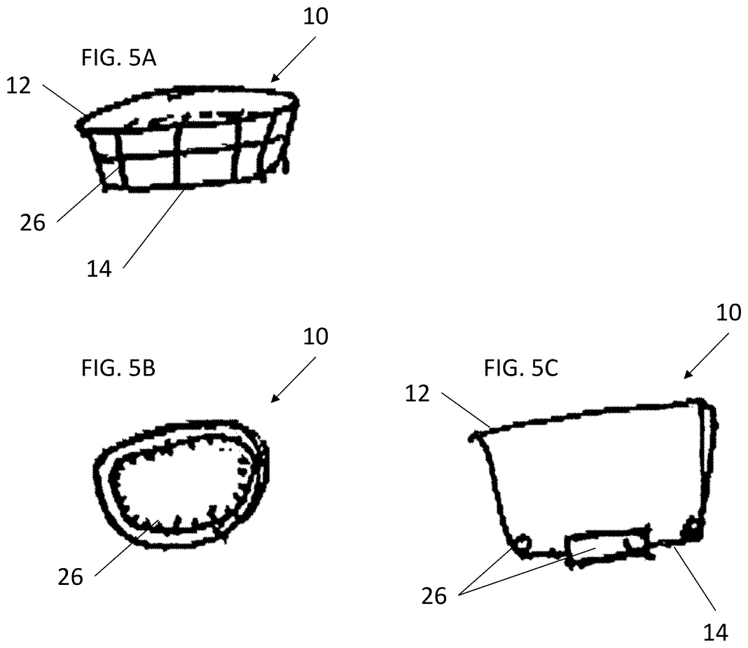

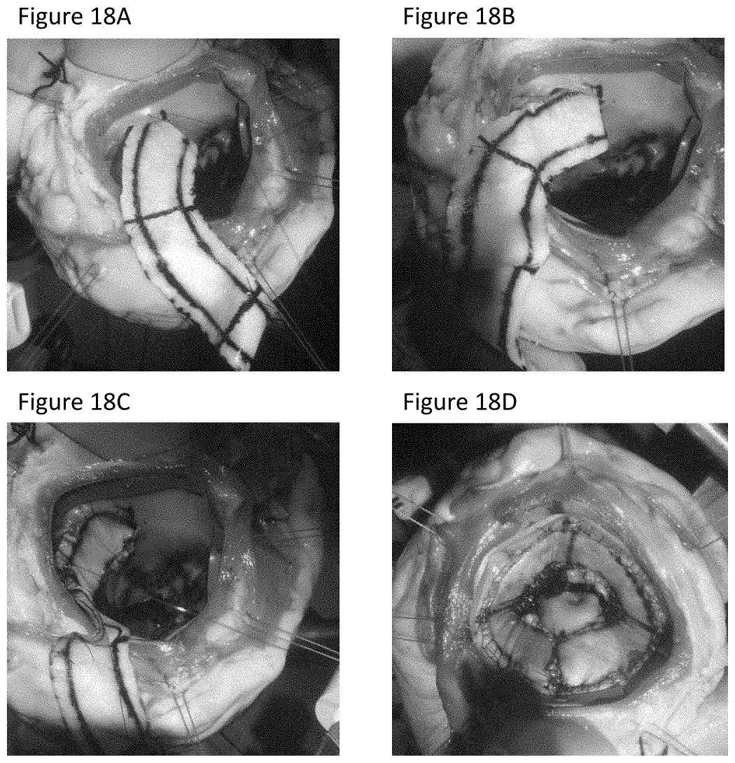

Referring now to FIG. 5A through FIG. 5C, in some embodiments device 10 can further include one or more sewing cuffs 26. Sewing cuffs 26 can be attached to the exterior of device 10, to annular edge 12, to apical edge 14, or combinations thereof. Sewing cuffs 26 provide device 10 with larger and more durable attachment surfaces for suturing to a subject's tissues. In some embodiments, device 10 can further include a small extension beyond annular edge 12 and apical edge 14 to increase attachment surfaces for sutures (e.g., FIG. 18A, 2 mm extensions marked by solid line parallel to annular and apical edges of the collar device).

Device 10 can be constructed from any material suitable for implanting, including but not limited to biocompatible polymers, fabrics, plastics, metals, as well as biological tissue such as autografts, allografts, xenografts, and engineered tissue constructs, and combinations thereof. Exemplary materials include Dacron® cloth (flexibility modified by albumin coating), gluteraldehyde-fixed bovine pericardium, a subject's native pericardium, and the like.

Materials including tissue can be can be treated with a sterilization step. The sterilization step can apply any suitable sterilization method, including but not limited to radiation (e.g., gamma radiation, x-ray radiation, ultraviolet sterilization, and electron beam processing), gaseous formaldehyde, carbon dioxide, ozone, ethylene oxide, peracetic acid, ethanol, hydrogen peroxide, and the like. The tissue can be provided with original cells, completely decellularized, or decellularized and reseeded with host cells. In some embodiments, the tissue can be enhanced with one or more additives, including but not limited to one or more additional extracellular matrix material and/or blends of naturally occurring extracellular matrix material, such as collagen, fibrin, fibrinogen, thrombin, elastin, laminin, fibronectin, vitronectin, hyaluronic acid, chondroitin 4-sulfate, chondroitin 6-sulfate, dermatan sulfate, heparin sulfate, vixapatin (VP12), heparin, and keratan sulfate, proteoglycans, and combinations thereof. The additives can include natural peptides, such as glycyl-arginyl-glycyl-aspartyl-serine (GRGDS), arginylglycylaspartic acid (RGD), and amelogenin. In some embodiments, the additives can include nutrients, such as bovine serum albumin. In some embodiments, the additives can include vitamins, such as vitamin B2, vitamin Ad, Vitamin D, Vitamin E, and Vitamin K. In some embodiments, the additives can include nucleic acids, such as mRNA and DNA. In some embodiments, the additives can include natural or synthetic steroids and hormones, such as dexamethasone, hydrocortisone, estrogens, and its derivatives. In some embodiments, the additives can include growth factors, such as fibroblast growth factor (FGF), transforming growth factor beta (TGF-β), and epidermal growth factor (EGF). In some embodiments, the additives can include a delivery vehicle, such as nanoparticles, microparticles, liposomes, viral and non-viral transfection systems. The additives can include one or more therapeutics. The therapeutics can be natural or synthetic drugs, including but not limited to: analgesics, anesthetics, antifungals, antibiotics, anti-inflammatories, nonsteroidal anti-inflammatory drugs (NSAIDs), anthelmintics, antidotes, antiemetics, antihistamines, anticancer drugs, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, fluorescent nanoparticles such as nanodiamonds, sedatives, sulfonamides, stimulants, sympathomimetics, tranquilizers, urinary anti-infectives, vasoconstrictors, vasodilators, vitamins, xanthine derivatives, and the like.

### Valve Translocation Kits

The present invention also encompasses surgical kits for translocating a valve. The kits can includes one or more devices 10, wherein each device 10 has the same size or a range of sizes to be selected by a surgeon to fit within a subject. The kits can further include one or more instruments relevant to the translocation procedure, including but not limited to: suture needles, suture thread, suture pledgets, forceps, scissors, scalpels, and the like. In some embodiments, the kits can further include one or more tools to measure portions of a subject's heart and to select dimensions of a device 10, such purpose-built sizers to measure a subject's native annulus and mitral valve circumference. In some embodiments, the kits can further include instructions for using a 3D echocardiogram to perform the measurements. For example, a 3D echocardiogram may be performed prior to an operation and a 3D analysis system may perform “in-silico” modeling to determine the optimal dimensions of a device 10.

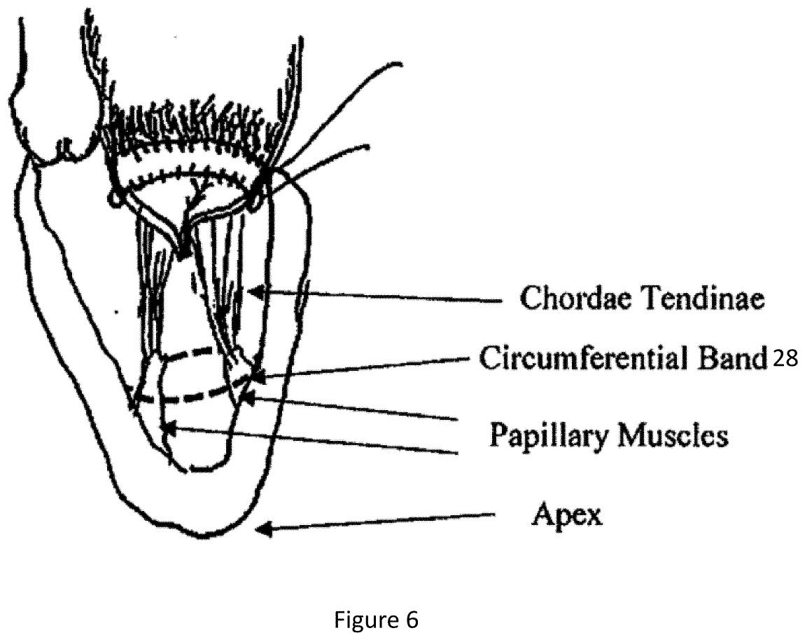

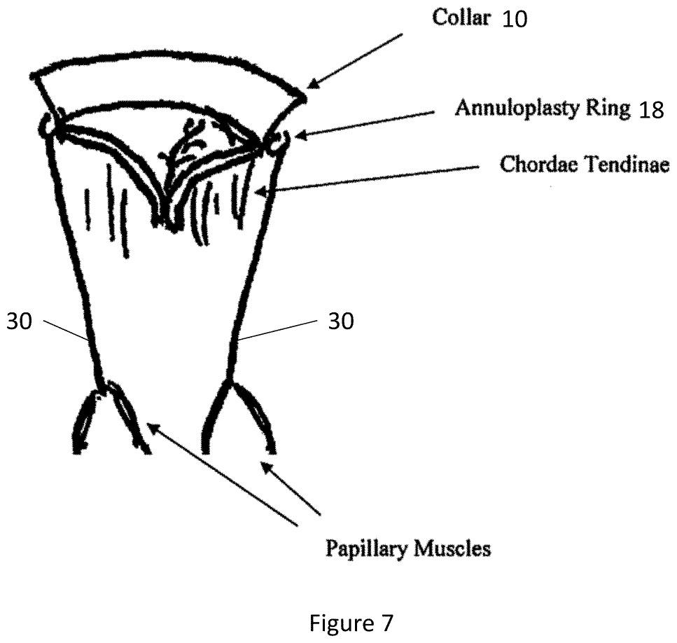

In some embodiments, the kit may also include other tools that further treat FMR. For example, the kit may include a circumferential band 28 configured to bring the papillary muscles closer together, such as by placing circumferential band 28 around the papillary muscles (FIG. 6). In another example, the kit may include one or more tethers 30 configured to increase and maintain the apical length or displacement of an implanted device 10 (FIG. 7). Tethers 30 can be attached to the papillary muscles at one end and to apical edge 14 of device 10 or to a large pledget secured to the epicardium of the heart. Tethers 30 can be constructed from ePTFE sutures, such as sutures that are commonly used for repair of degenerative mitral regurgitation. In another example, the kit may include additional cuffs 26 attachable to device 10 and configured to further reduce the opening diameter of annular edge 12 or apical edge 14. Cuffs 26 each have a cuff aperture and an outer diameter that attaches, such as by suturing, to annular edge 12 or apical edge 14 of device 10, and the subject's anatomy can be sutured to the cuff aperture.

### Methods of Valve Translocation

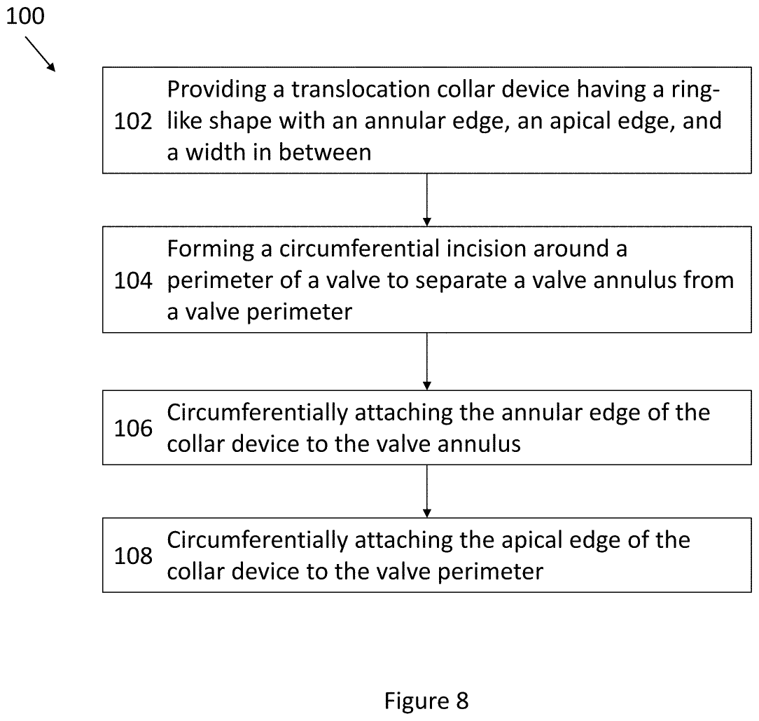

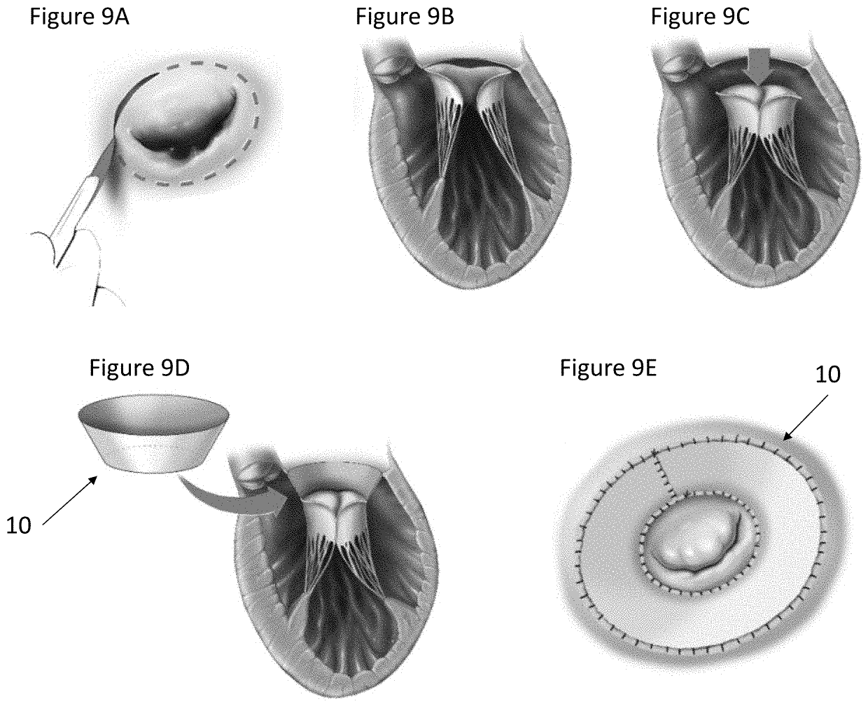

The present invention further includes methods of using the translocation collar devices of the present invention. Referring now to FIG. 8, an exemplary method 100 is depicted. Method 100 begins with step 102, wherein a translocation collar device of the present invention is provided, the collar device having a ring-like shape with an annular edge, an apical edge, and a width in between. In step 104, a circumferential incision is formed around a perimeter of a valve to separate a valve annulus from a valve perimeter. The incision keeps the structure of the original valve leaflets, commissures, and other physical features intact, including the chordae tendinae and associated muscles (FIG. 9A through FIG. 9C). In step 106, the annular edge of the collar device is circumferentially attached to the valve annulus. In step 108, the apical edge of the collar device is circumferentially attached to the valve perimeter (FIG. 9D, FIG. 9E). The attachment means can include sutures, adhesives, staples, and the like.

In some embodiments, the dimensions of the valve are sized before a translocation collar device is provided. In one embodiment, the dimensions are sized by measuring the native annulus after circumferential incision and detachment of the valve with a first set of sizers, and measuring the circumference of the valve perimeter with a second set of sizers. In another embodiment, the dimensions are sized by measuring the heart with a 3D echocardiogram and analyzing the measurement with a 3D analysis system. In one embodiment, the annular edge is sized to a subject's annulus. The dimensions of the apical edge can be reduced or “downsized” based on the size of the annulus edge. For example, the apical edge can be preset to be one or more sizes smaller than the annular edge, as would be understood by those skilled in the art. For example, an apical edge can be 2 sizes smaller than the annular edge (in which 1 size=5 mm smaller circumference, 2 sizes=10 mm smaller circumference). For reference, a common annular edge size is size 38 (having an orifice area of about 722 mm², circumference of about 95 mm). Reducing size 38 by two 2 sizes results in a smaller apical edge of size 34 (having an orifice area of about 572 mm², circumference of about 85 mm). In some embodiments, the sizes can be labeled as extra small, small, medium, large, extra large, and the like. The annular edge of each size can be based on the label, and the apical edge of each size can be preset as the circumference of the annular edge reduced by a set amount (such as 10 mm).

In some embodiments, the valve annulus and valve perimeter are fully separated prior to attaching the collar device. In some embodiments, the valve annulus and valve perimeter are partially separated, and the collar device is attached as the circumferential incision is made in a stepwise sequence. A stepwise sequence can be advantageous in that the valve annulus and valve perimeter are always attached together, either by natural tissue or by the collar device, improving valve stability throughout the operation. For example, the method can include steps for marking the valve to prevent rotation of valve leaflets relative to the annulus. The method can include a step of marking the valve in portions, such as thirds or quadrants. The method can further include a step of separating the valve annulus from the valve perimeter a portion at a time, such as a third at a time or a quadrant at a time. For each portion that is separated, one or more (e.g., three) horizontal mattress sutures can be placed between the valve annulus and the annular edge of the collar, as well as between the valve perimeter and the apical edge of the collar. The sutures allow the collar to be seated below the plane of the annulus and the valve leaflets. Each portion can be sutured with a running suture. After a portion is separated and sutured to the collar, a succeeding portion is separated and sutured to the collar, until all portions have been separated and sutured to the collar.

In some embodiments, method 100 is performed percutaneously. The collar device can be introduced near the valve site, such as in a ventricle or atrium, using a transapical technique, a transfemoral technique, a transaortic technique, a transseptal technique, and the like to implant the collar device in a circumferential fashion around a subject's normal valve annulus.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out exemplary embodiments of the present invention, and are not to be construed as limiting in any way the remainder of the disclosure.

### Example 1: In Vivo Swine Analysis

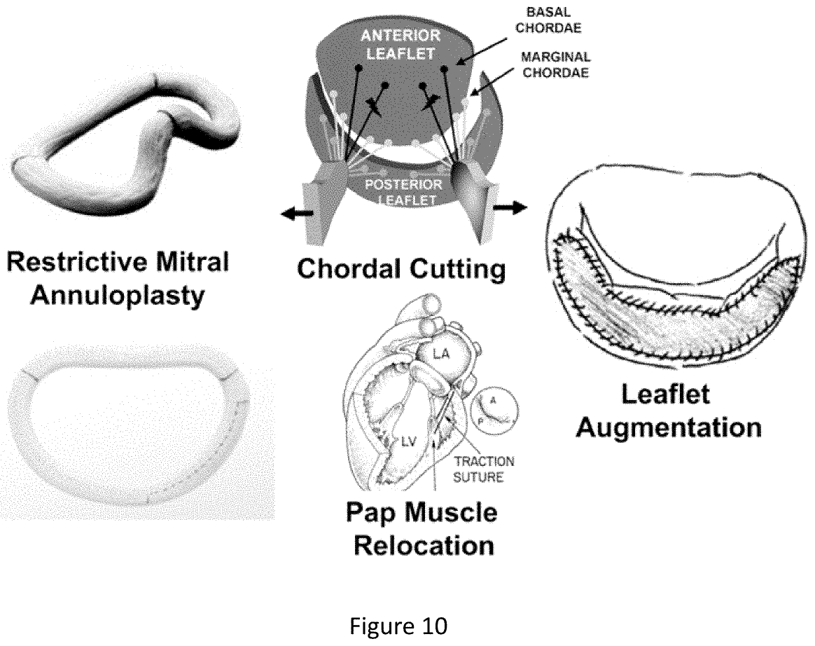

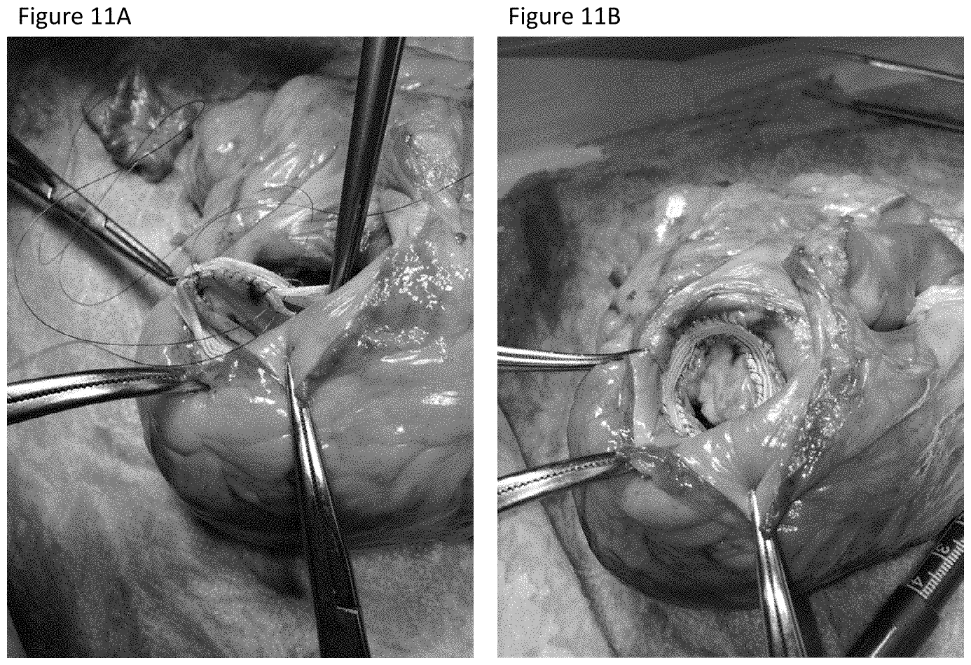

There is no reliable and durable mitral valve repair option for patients with functional mitral regurgitation (FMR). Existing devices (tissue or mechanical) and methods are not durable, suffer high recurrence rates, and are limited by increased risk of bleeding, prosthetic valve dysfunction, infection, and thromboembolism (FIG. 10). The present study aims to improve upon valve repair by examining the efficacy of valve translocation collars.

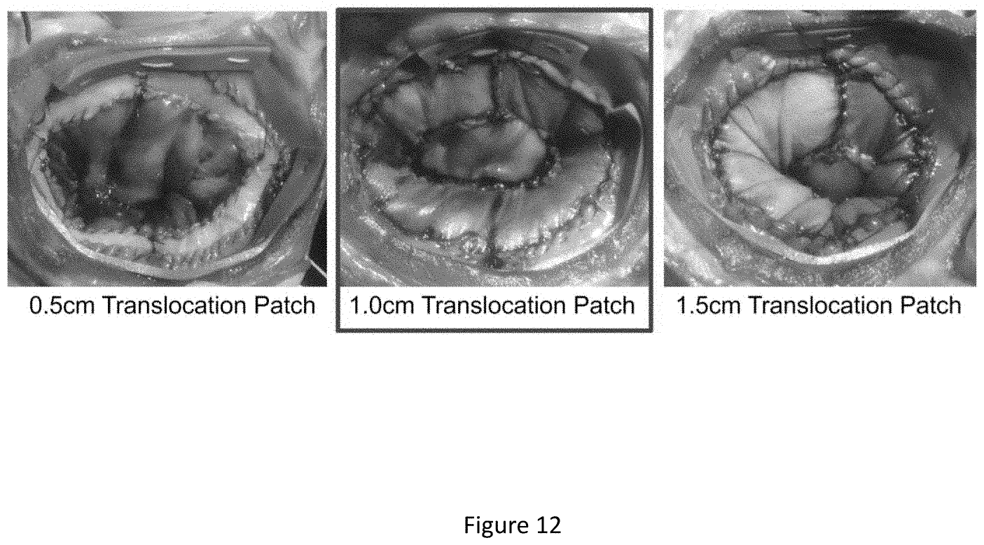

Yorkshire swine (50-70 kg) were placed on cardiopulmonary bypass for translocation patch repair (n=7). Inner patch diameter was sized to anterior mitral valve leaflet. Leaflet was detached from the annulus and the bovine pericardial patch was sewn in. Pre- and post-operative echocardiography were used to evaluate efficacy of patch (FIG. 12A).

The collar implants improved coaptation from 0-4 mm to 6-10 mm (FIG. 12B). Other areas of improvement include: improved predictors of repair durability; tenting area reduced; leaflet angles improved. Mild suture line regurgitation was observed in 3/7 swine. Mitral valve area after repair was about 2.3 cm² (normally 5 cm²).

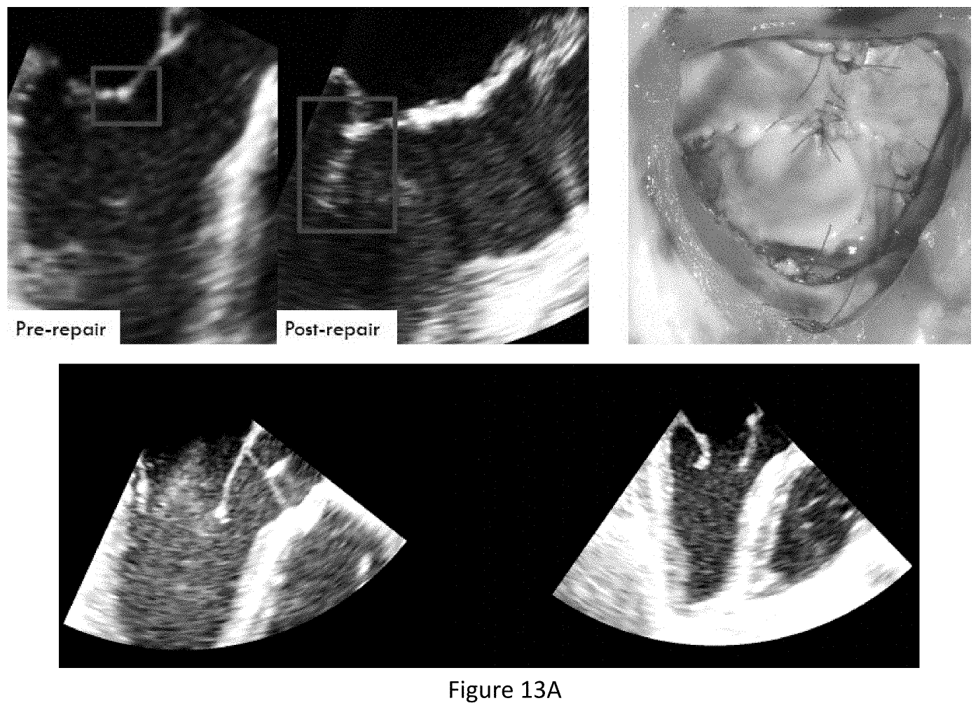

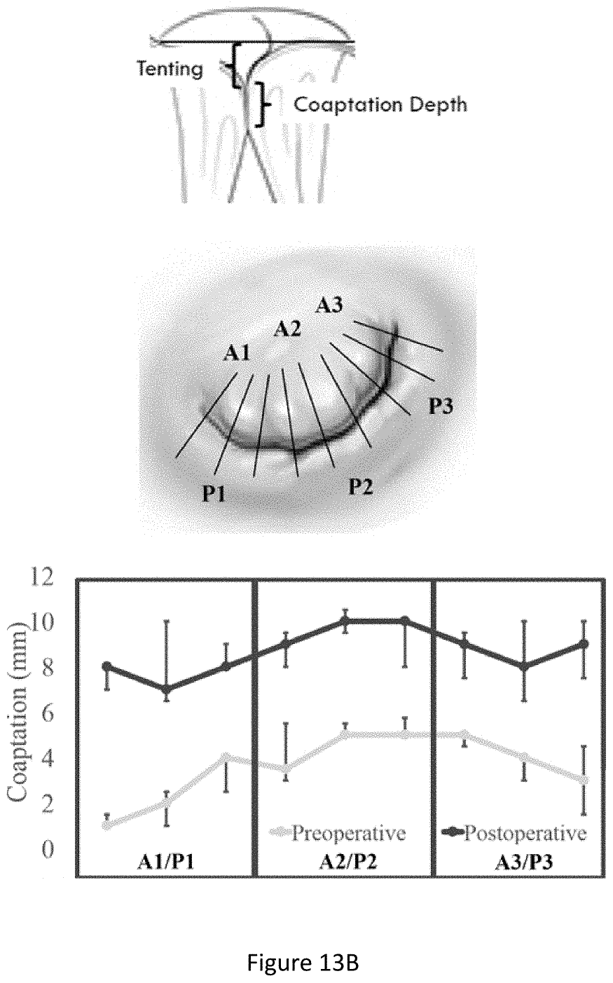

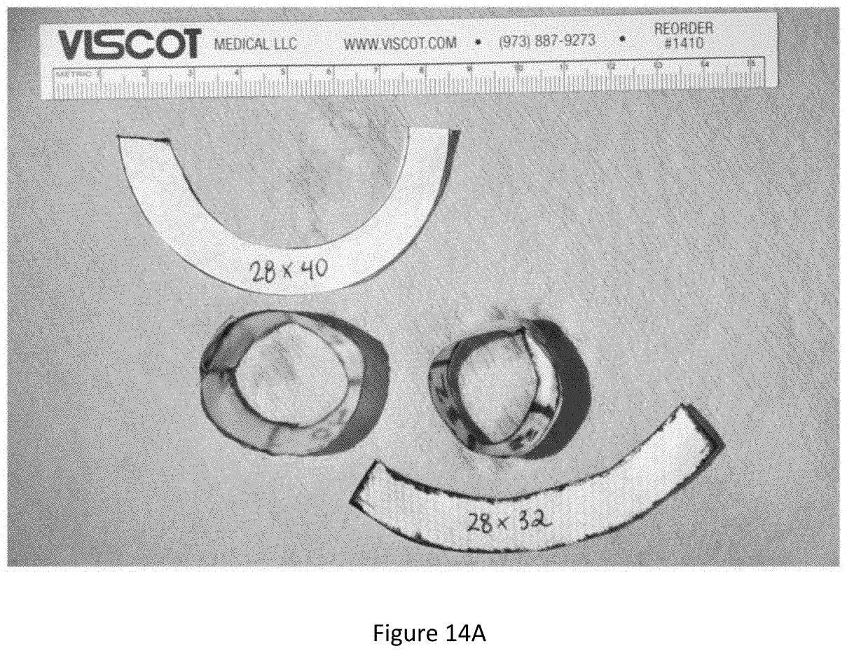

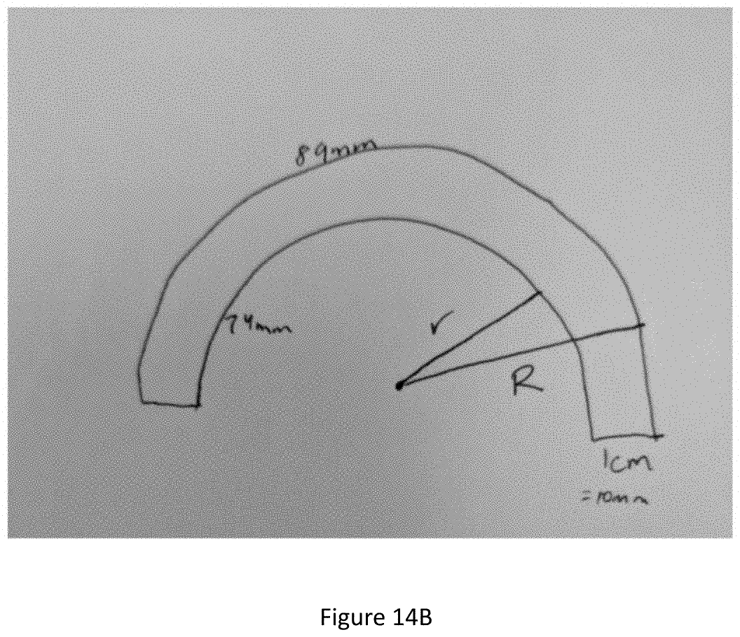

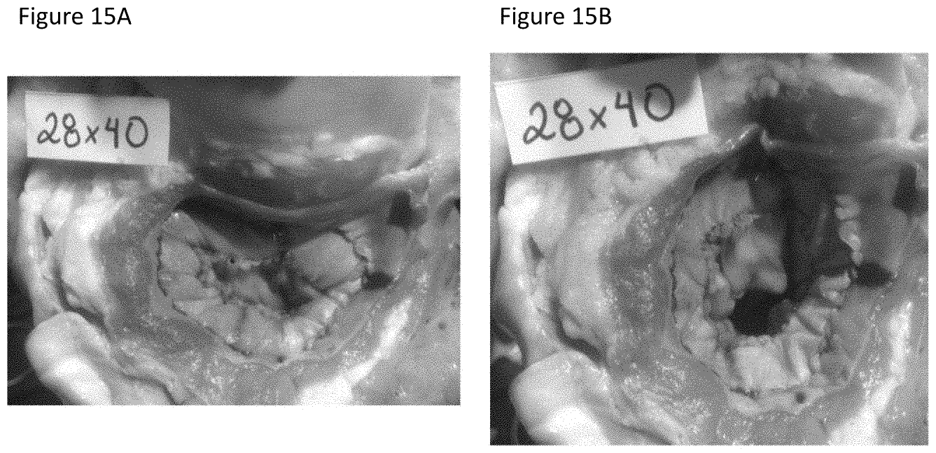

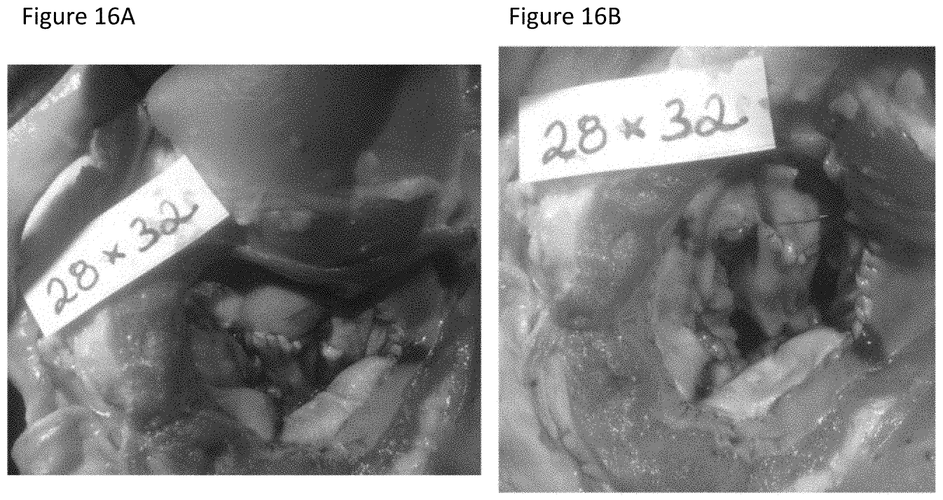

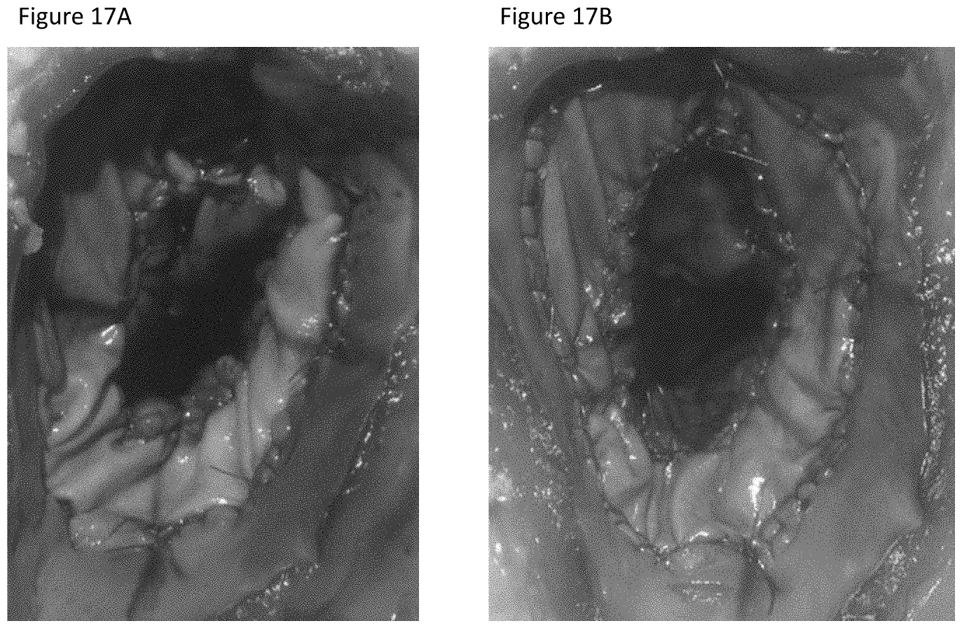

Modifications were made to reduce or prevent suture line regurgitation. The outer diameter of the collar implant is sized to fit the dimensions of the patient's annulus, forming a smaller patch diameter having a more acute patch angle (FIG. 13A). Locking sutures are used to prevent crimping (FIG. 16A, FIG. 16B). 2 mm tabs were added to the upper and lower edges of the collar to improve suturing, and horizontal mattress sutures were used to seat the collar below the annulus/leaflet (FIG. 17A through FIG. 17D).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A translocation collar device comprising:
a substantially ring-shaped band of material having an annular edge, an apical edge opposite from the annular edge, and a width in between;
wherein the collar device is attachable to a circumferentially separated valve such that the annular edge is attached to a valve annulus and the apical edge is attached to a valve perimeter to translocate the valve in an apical direction.

2. The device of claim 1, wherein the annular edge has a diameter between about 20 mm and 60 mm.

3. The device of claim 1, wherein the apical edge has a diameter between about 5 mm and 15 mm.

4. The device of claim 1, wherein the width is between about 5 mm and 15 mm.

5. The device of claim 1, wherein the width is biased such that the annular edge and the apical edge are separated by a variable distance.

6. The device of claim 1, wherein the device is constructed from a length of material having an arc shape, with a first end, a second end, an outer edge having a length equal to a circumference of the annular edge, and an inner edge having a length equal to a circumference of the apical edge, such that the first end and the second end are joinable together to form a substantially ring-shaped band.

7. The device of claim 1, further comprising one or more concentric folds aligned in parallel with the annular edge and the apical edge, such that the width is variable.

8. The device of claim 7, wherein the width is fixable by applying one or more sutures or adhesives to the one or more concentric folds.

9. The device of claim 1, further comprising one or more annuloplasty rings attached to the annular edge, the apical edge, or both.

10. The device of claim 1, further comprising one or more cuffs attached to the annular edge, the apical edge, or a position in between.

11. The device of claim 1, wherein the material is selected from the group consisting of: polymer, fabrics, plastics, metals, autograft tissue, allograft tissue, xenograft tissue, and engineered tissue constructs.

12. A method of translocating a valve, the method comprising the steps of:
providing a translocation collar device having a ring-like shape with an annular edge, an apical edge, and a width in between;
forming a circumferential incision around a perimeter of a valve to separate a valve annulus from a valve perimeter;
circumferentially attaching the annular edge of the collar device to the valve annulus; and
circumferentially attaching the apical edge of the collar device to the valve perimeter.

13. The method of claim 12, wherein the translocation collar device is sized to fit the valve annulus and valve perimeter by measuring the dimensions of the valve annulus and valve perimeter.

14. The method of claim 12, wherein the translocation collar device is sized to fit the valve annulus and valve perimeter by performing and measuring the dimensions of a 3D echocardiogram of a heart containing the valve.

15. The method of claim 12, wherein the circumferential incision is formed while keeping the valve and associated structures intact, the associated structures including leaflets, commissures, chordae tendinae, and papillary muscles.

16. The method of claim 12, wherein the annular edge and the apical edge of the collar device are attached after the circumferential incision is fully formed and the valve annulus is completely separated from the valve perimeter.

17. The method of claim 12, wherein the annular edge and the apical edge of the collar device are attached as the circumferential incision is being formed and the valve annulus is partially separated from the valve perimeter.

18. A valve translocation kit, comprising:
at least one translocation collar device having a ring-like shape with an annular edge, an apical edge, and a width in between; and
one or more suture threads, suture pledgets, forceps, scissors, scalpels, and combinations thereof.

19. The kit of claim 18, further comprising one or more circumferential bands, each circumferential band configured to wrap around papillary muscles connected to a valve.

20. The kit of claim 18, further comprising one or more tethers, each tether configured to attach to the apical edge of device at a first end and papillary muscles connected to a valve at a second end.
